(19) United States
(12) Patent Application Publication
(10) Pub. No.: US 2024/0091376 A1
(43) Pub. Date: Mar. 21, 2024

(54) ALZHEIMER'S DISEASE TREATMENT AND METHODS

(71) Applicant: Pharmzandia Corporation, Henderson, NV (US)

(72) Inventor: Peter Butzloff, Walnut, CA (US)

(21) Appl. No.: 18/239,131

(22) Filed: Aug. 29, 2023

Related U.S. Application Data
(63) Continuation of application No. PCT/US2022/017589, filed on Feb. 23, 2022.
Application No. PCT/US2022/017589 is a continuation of application No. PCT/US2021/062908, filed on Dec. 10, 2021.
Application No. PCT/US2021/062908 is a continuation of application No. PCT/US2021/063977, filed on Dec. 17, 2021.
Application No. PCT/US2021/063977 is a continuation of application No. PCT/US2021/062908, filed on Dec. 10, 2021.
(60) Provisional application No. 63/154,899, filed on Mar. 1, 2021.
(60) Provisional application No. 63/154,899, filed on Mar. 1, 2021.

(51) Int. Cl. A61K 47/69; A61K 47/54; A61K 47/60

(52) U.S. Cl. CPC A61K 47/6929; A61K 47/54; A61K 47/60

(57) ABSTRACT

A nanoparticle composed of buckminsterfullerene bonded to glutamine, gamma amino butyric acid (GABA) and adenosine triphosphate is provided. This nanoparticle helps to regulate the energy metabolism, neural excitability, and signal transduction within the synapse and between the astrocytes and the neuronal networks of the human brain when these become dysfunctional. It is intended as a treatment for Alzheimer's disease, Lewy Body disease and other neuropathological diseases of the glutamine-glutamate-GABA cycle. Utility for remediating comorbid sexual dysfunction is promoted, as well as the antioxidant and protein oligomer disassembly properties. The adenosine triphosphate adduct provides a reversible inorganic phosphate energy storage and supply, the glutamine adduct provides a stable reservoir of this amino acid that is resistant to breakdown by reactive oxygen species, reactive nitrogen species, and astrocytes. The GABA and buckminsterfullerene functional groups are anti-oxidants to treat the most oxidatively stressed regions at neural structures.

### CROSS-REFERENCE TO RELATED APPLICATIONS

This application is a continuation of international application PCT/US22/17589 which claims the benefit of international application PCT/US21/62908 filed on Dec. 10, 2021 which claims the benefit of U.S. provisional patent application No. 63/154,899 filed Mar. 1, 2021; PCT/US22/17589 also claims priority from U.S. provisional patent application No. 63/154,899 and claims the benefit of international application PCT/US21/63977 filed on Dec. 17, 2021 which claims the benefit of international application PCT/US21/62908.

### BACKGROUND OF THE INVENTION

### Field of the Invention

The invention provides a composition of matter that relates generally to the field of neurological disease treatment, to avoid and substantially repair neurocognitive damage associated with imbalances in the glutamine-glutamate and glutamine-gamma amino butyric acid (GABA) cycles as mediated by glial cells at the neural junctions of the human brain. Fullerene molecules serve as an antioxidant and also as a carrier for neurotransmitters required to establish multifunctional cognitive homeostasis in Alzheimer's Disease (AD) and related neurological pathologies such as Lewy-body disease (LD) and amyotrophic lateral sclerosis (ALS).

### Description of the Prior Art

Alzheimer's Disease, a type of dementia, is the most fatal non-curable neurodegenerative disease. AD affected more than 57 million people globally as reported in 2019 and is estimated to double every two decades to affect 153 million people by the end of 2050. The growth in the number of individuals living with dementia underscores the need for interventions and public health policies worldwide to address this expected increase. Modifications to healthcare and planning will be needed to accommodate the associated long term economic ramifications for the impact of AD to both families with aging members and the economic well-being of entire nations who must allocate ever more resources to care for increasing numbers of people with dementia who are unable to care for themselves.

Glutamatergic dysfunction of the cycle between glutamine and glutamic acid is characterized by the loss of the glutamatergic synapses, leading to the clinical symptoms of AD in patients. This process is now thought by some researchers to be a more primary feature of AD. The tripartite synapse is where glutamatergic neurotransmission occurs. The tripartite synapse is where astrocytes can become pathologically altered in their function to control the amount of calcium ions, the amount of the neurotransmitter gamma amino butyric acid (GABA), and the ratio of glutamine (GLN) to glutamic acid (GLU) at the synaptic region of the neuron.

There are no extracellular enzymes to degrade glutamate in the extracellular space between the synaptic terminals. Therefore, glutamatergic dysfunction represents an underappreciated therapeutic target for the treatment of AD. Normally, the uptake of extracellular glutamate by one of five known sodium dependent excitatory amino acid transporters (EAATs), of which EAAT2 is the major transporter of glutamate out of the extracellular space. The EAATs are located mostly in the astrocytes in the adult human brain. If the astrocytic production of EAATs become deficient, this causes a hyper-glutamatergic state associated with chronic brain inflammation, leading to the effects of AD as well as other neurocognitive dysfunctions.

Excessive glutamate uptake in astrocytes is associated with the production of damaging reactive oxygen species (ROS) and reactive nitrogen species (RNS) such as nitric oxides (NO), which can lead to the formation of salt-bridges between oxidized proteins such as tau and beta amyloid. The amyloid plaque burden or neurofibrillary tangles are another characteristic hallmark of AD, but the state-of-the-art drugs and medications that have been developed to remove these oligomers have been shown to be ineffective in producing an improvement of the clinical AD symptoms. Therefore, AD and similar dementia must have a root cause wherein the neurofibrillary protein tangles are one of the complicating results. The medical community is forced to look elsewhere for new research and possible treatment at this time.

The neuron to astrocyte interaction via the glutamine to GABA cycle is critical for the replenishment of the neuronal glutamate pool for neurotransmission. What has never been clarified, however, is how to aid in the regulation of energy metabolism, neural excitability, and signal transduction within and between the astrocytes and the neuronal networks when these become disrupted or dysfunctional in AD and other neuropathological diseases. The present invention provides such a regulatory aid in the form of specially designed nanoparticles to re-establish homeostatic neuronal and astrocytic regulation to promote prevention, treatment, and recovery from the glutamate-glutamine cycle dysfunction in Alzheimer's disease.

### SUMMARY

This invention is a nanoparticle composed of buckminsterfullerene (C60) bonded to glutamine (GLN), gamma amino butyric acid (GABA), and an adenosine phosphate functional group such as adenosine triphosphate (ATP). This nanoparticle ensemble is designed to regulate the energy metabolism, neural excitability, and signal transduction within the synapse and between the astrocytes and the neuronal networks of the human brain when these become dysfunctional. It is intended to be used as a treatment for Alzheimer's disease, Lewy Body disease and other neuropathological diseases of the glutamine-glutamate-GABA cycle. The adenosine triphosphate (ATP) adduct provides a reversible inorganic phosphate energy storage and supply, the glutamine (GLN) adduct provides a stable reservoir of this amino acid that is resistant to breakdown by ROS and RNS, and at least one GABA adduct provides a primarily inhibitory synaptic regulator. The antioxidant properties of the functional groups are deliberately carried to the most oxidatively stressed region at neural structures, being the presynaptic terminal where glutamine deficits may appear, while also providing a storage reservoir of reducing hydrogen protons on the C60 and the amine functionality of GABA to confer a localized chemical reducing condition to this compound.

In a key aspect, C60-GLN-GABA-ATP and its metabolites provide an artificial pathway to supplement and accelerate the trafficking of cations for proton exchange to prevent or remove salt accumulation among oligomeric tau and beta amyloid fibrils. This function acts to disassemble the oligomeric plaques formed by salt cations by extracting these cations, so that they may not serve as salt bridges. C60 is normally considered anionic when it collects as many as six negative charges, however it may also attract and store as many as 5 hydrogen protons, in which both hydrogen bonding as well as aromatic pi to cationic pi bonding contributes to the stability of these structures and defines how this collective ensemble serves to traffic both protons and physiological cations such as potassium and sodium.

In a related aspect, the C60-GLN-GABA-ATP and its metabolites accrues and transports hydrogen protons to regions removed from the mitochondria where protons are required to exchange for physiological cations such as potassium, and sodium. This aspect can supplement endogenous substances fulfilling the same role.

In another related aspect, the uptake of extracellular glutamate by one of five known sodium dependent excitatory amino acid transporters (EAATs), of which EAAT2 is the major transporter of glutamate out of the extracellular space, is performed by C60-GLN-GABA-ATP and its metabolites. This becomes important when the astrocytic production of EAATs become deficient, and directly avoids the formation of a hyper-glutamatergic state associated with chronic brain inflammation, thereby treating the effects of AD as well as other neurocognitive dysfunctions.

In another related aspect, the glutamine functional group bonded to the C60 stabilizes conditionally essential glutamine to remediate local glutamine deficits in brain tissues against upregulated astrocyte conversion of glutamine to glutamate, thereby restoring synaptic glutamine homeostasis.

In another aspect, the technological hurdle of supplying exogenously produced GABA neurotransmitter to the brain is provided by using a buckminsterfullerene (C60) carrier to enable crossing of the blood brain barrier and allow GABA's well known medical benefits, including reducing blood pressure and enhancing long-term memory, to be directly promoted to each brain region and all brain tissues.

In a related aspect, the normalization of blood pressure and the phosphate ion shuttling effect of the C60 with adenosine phosphate promotes the needed energetics to remediate sexual dysfunction, which is common in Alzheimer's and Lewy body disease patients. In males, this sexual dysfunction is termed erectile dysfunction; correction of this medical issue is short term, spontaneous, voluntary, and will become efficacious within 4 hours after administration.

In a related aspect, the buckminsterfullerene functional group has well known antimicrobial properties. These properties allow the C60-GNL-GABA-ATP composition to treat, delay or arrest the incidence of microbial infections in the brain, wherein the nano-aerosol formulation can expedite targeted delivery to the brain by avoiding a passage through the digestive system.

In a related aspect, the transport of GABA into the brain by C60 allows it to be protected by the C60 functional group so that this form of GABA is unable to be easily broken down by neural enzymes, especially those released by astrocytes. This enhanced stability promotes the circulation of GABA with an extended lifetime or residence, in which it acts as both an antioxidant and as a critically important neurotransmitter.

In yet another aspect, C60-GLN-GABA-ATP and its metabolites disrupts sodium ion salt bridges between beta amyloid plaque fibrils to return individual amyloid monomers to their proper conformation and neurological function. Largely, it is the presence of the C60 molecule being tethered to three neurotransmitter functional groups that helps to bring it to where each of the natural (monomeric) neurotransmitters reside, to then disassemble detrimental salt bridges between the tangled protein agglomerates. The high negative charge density acquisition of the C60 group enables the abstraction and sequestering of sodium cations onto itself and away from the plaque proteins.

In a related aspect of C60-GLN-GABA-ATP nanoparticle chemistry, the function of each of the tethered neurotransmitter functional groups provides a similar neurotransmitter capability and function to that of each of the natural (monomeric) neurotransmitters GLN, GABA, and ATP used to synthesize it. Therefore, the dearth or absence of any one of the natural neurotransmitters in monomeric form, will be supplemented in function by its respective adduct in conjunction with the protective antioxidant C60 functional group. In promoting normal neurological homeostasis, the root cause of neural inflammation is therefore addressed, and clearance of the extracellular fluid of detritus and misfolded proteins can again resume.

In various aspects of the delivery of C60-GNL-GABA-ATP to the human brain, methods of inhalation of a vapor containing the nano-aerosol, of oral consumption, and of liquid infusion by droplets placed onto the eye are exemplary embodiments of administration of this nanoparticle composition to the consumer or patient.

These and other advantages of the present invention will be further understood and appreciated by those skilled in the art by reference to the following written specification, claims, and appended drawings.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### BRIEF DESCRIPTION OF DRAWINGS

FIG. 1 is an illustration of the molecular structures of raw materials used to make the triple neurotransmitter composition.

FIG. 2 is an illustration of the molecular structures of a reversible mineral phosphate reaction between ATP and ADP.

FIG. 3 is an illustration of the molecular structures of products from a reaction between C60 and ATP.

FIG. 4 is an illustration of the molecular structures of products from a reaction between C60 and a mineral phosphate.

FIG. 5 is an illustration of the molecular structures of products from a reaction between C60, GLN, and GABA.

FIG. 6 is an illustration of the molecular structures of a reaction between C60-GLN-GABA and ATP to form the triple neurotransmitter C60-GLN-GABA-ATP.

FIG. 7 is an illustration of molecules of triple neurotransmitter C60-GLN-GABA-ATP at neurons afflicted with Alzheimer's disease oligomers.

FIG. 8 is a chart of an exemplary synthesis of C60-GLN-GABA-ATP formulated for a nano-aerosol administration.

FIG. 9 is a chart of an exemplary synthesis of C60-GLN-GABA-ATP formulated for oral administrations.

FIG. 10 is an illustration of the personal administration of a nano-aerosol formulated C60-GLN-GABA-ATP.

FIG. 11 is an illustration of the FTIR experimental test data for GLN

FIG. 12 is an illustration of the FTIR experimental test data for C60-GLN.

FIG. 13 is an illustration of the FTIR experimental test data for C60-ATP.

FIG. 14 is an illustration of the FTIR experimental test data for GABA.

FIG. 15 is an illustration of the FTIR experimental test data for C60-GABA.

FIG. 16 is an illustration of the FTIR experimental test data for C60-GLN-GABA.

FIG. 17 is an illustration of the FTIR experimental test data for C60-GLN-GABA-ATP.

FIG. 18 is an illustration of experimental negative mode mass spectrograph data for C60.

FIG. 19 is an illustration of experimental negative mode MALDI-TOF mass spectrograph data for C60-GLU-GABA-ATP.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### DETAILED DESCRIPTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

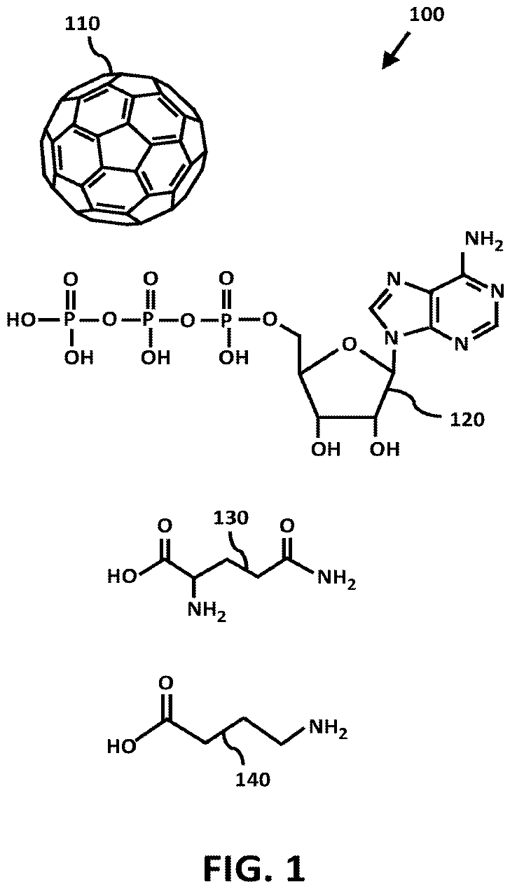

FIG. 1 illustrates the molecular structures, 100 used to synthesize the nanoparticle compositions of the present invention. Buckminsterfullerene 110 is a single molecule comprised of 60 carbon atoms arranged as a sphere and having the chemical formula of C60. The neurotransmitter adenosine tri-phosphate (ATP) 120 has the chemical formula C₁₀H₁₆N₅O₁₃P₃. The molecular structure of ATP 120 comprises an adenine attached by a nitrogen atom to ribose (a sugar) which in turn is attached a triphosphate group. The three labile phosphate groups are the source of biochemical energy in all living cells and is used to propagate nerve impulses along neurons. Glutamine (GLN) 130 has formula C₅H₁₀N₂O₃ and is one of the few amino acids that can directly cross the blood brain barrier (BBB). GLN 130 is reversibly metabolized by astrocytes to form glutamate. GLN 130 operates together with ATP 120 to release biochemical energy for cells. The neurotransmitter gamma aminobutyric acid (GABA) 140 has the chemical formula C₄H₉NO₂. GABA 140 is a major inhibitory neurotransmitter synthesized and delivered by GABAergic neurons. GABA 140 has seen limited usefulness as a supplement because of very poor blood brain barrier diffusion from outside the brain. From within the brain, GABA is subject to intensive breakdown by astrocyte GABA transaminase. Substances 110, 120, 130, 140 may be used to help create, process, or deliver parts of the composition of C60-GLN-GABA-ATP.

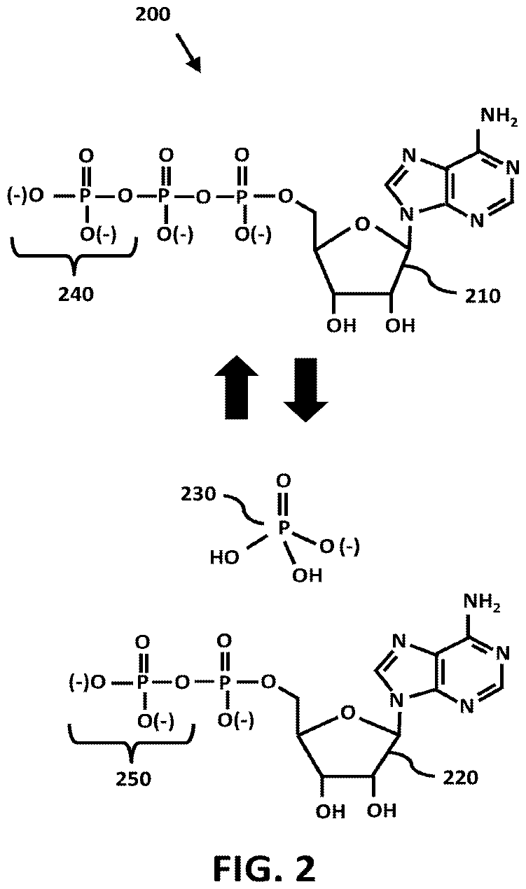

FIG. 2 illustrates the molecular structures of the reversible biochemical phosphate reactions 200 of the neurotransmitter adenosine tri-phosphate (ATP) 210. The adenosine triphosphate 210 reversibly disassociates into adenosine diphosphate (ADP) 220, with the loss of one free inorganic mineral phosphate group 230, where this reaction is shown by the downward direction of the heavy black arrow. ADP 220 and a mineral phosphate 230 can again become bonded to the ATP 210, where this reaction is illustrated by the upward direction of the heavy black arrow, to form the phosphate group 230 on ATP 210 as illustrated by the region bracketed by 240. This chemical process is part of the chemical respiration of the cell at physiological pH. It is understood that ADP 220 may also lose one more phosphate group 250 to generate adenosine monophosphate (AMP) in a similarly reversible manner. The AMP and the related cyclic adenosine monophosphate (cAMP) structures are sufficiently well understood as reversible metabolites of ADP and ATP and will react in like manner when used as a functional group of a nanoparticle such as C60-ATP, according to these teachings.

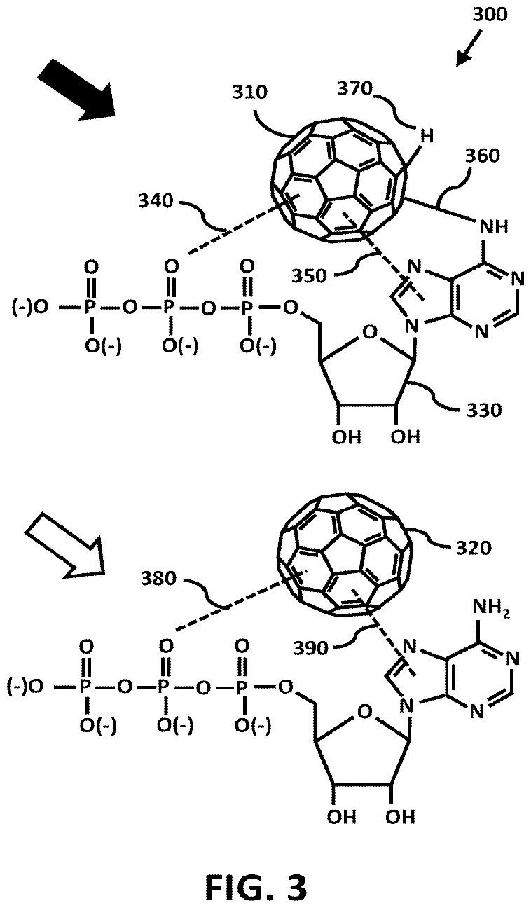

FIG. 3 illustrates buckminsterfullerene adenosine triphosphate (C60-ATP) synthesis 300. The reaction of the neurotransmitter ATP with carbon fullerene uses C60 310 to form the C60-ATP adduct 320. The amine functional group of adenosine triphosphate (ATP) 330 may form two aromatic pi-pi stacking bonds 340, 350 and a covalent bond 360 at the amine nitrogen with a transient hydrogen adduct 370 at neutral pH to form fullerene C60-ATP 320, where the product of this synthesis reaction shown by the direction of the large black arrow is favored above 55° C. However, ATP 330 may also form fullerene C60-ATP 320 where the product of this synthesis reaction is shown by the direction of the large white arrow, forming two aromatic pi-pi stacking bonds 380, 390 which is more favored without an amine reaction below about 55° C. It is understood that metabolites of the fullerene adenosine phosphate nanoparticles will be reversibly oxidized and reduced by the gain or loss of phosphate groups in the manner illustrated for ATP 210 in FIG. 2 in the context of this and the related adenosine phosphate fullerene compositions herein.

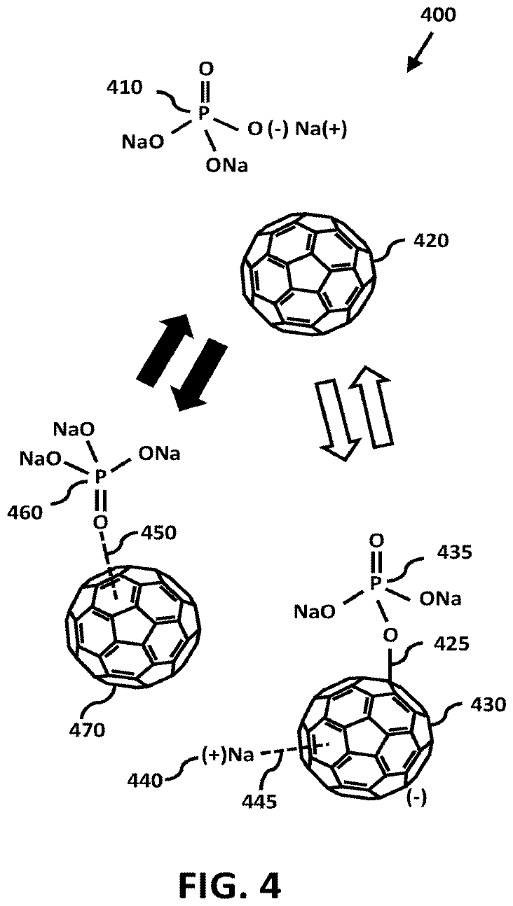

FIG. 4 illustrates the molecular structures for a C60 fullerene phosphate synthesis 400. The inorganic trisodium phosphate 410 from ATP or ADP is provided with three negative charged oxygen atoms that have counter-charged cations such as sodium which can become attracted to react with a C60 fullerene molecule 420. The fullerene may comprise, for example, a C60 carbon cage molecule, which can accept up to six electrons. It is understood that multiple mineral sodium phosphate groups may reversibly react with C60 420 to create fullerene phosphates, where the reaction may proceed in the direction of the white arrows to form a transient oxygen covalent bond 425 between C60, 430 and sodium phosphate 435, along with a sodium ion 440 that can become pi-cation 445 associated with C60 430. However, the reaction may also reversibly proceed in the direction of the black arrows to form a phosphonyl-pi bond as indicated by the dashed line 450 to stabilize the structure between the double bonded oxygen of phosphate 460 and C60 470. Shuttling of inorganic phosphates as well as of sodium is widely utilized in the human body in ATP, ADP, and AMP. Such phosphates assist with charge transport and shuttling in the electron transfer chain and the proton (H+) accumulation process using ATP synthase (ATPase) of cellular respiration at mitochondria. Medical evidence clearly points to a deficit in this type of shuttling for a wide range of neurological disorders including the energy deficit expressed in Alzheimer's disease. C60 can help to anchor ionic species by van-der-Waals attraction to biological cellular structures between organelles inside cells, as well as to the organic peptides within cellular organelles such as the mitochondria where respiration takes place. The presence of both inorganic phosphate and organic C60 functionality improves the cellular respiration process and overcomes some compatibility issues with transport of phosphate through oligomeric beta amyloid tangles and malformed peptides and neuropeptides to help with energy production in the mitochondria of the brain. This C60 mediated phosphate ion shuttling, along with the electron transport of the C60 functional group, significantly promotes the treatment of biochemical energetic dysfunction as an important part of remediating the pathology of Alzheimer's disease's neurological deficits.

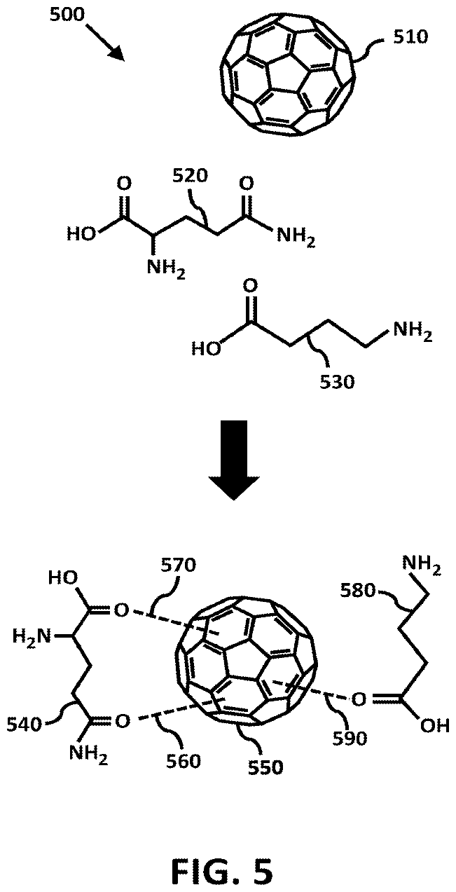

FIG. 5 illustrates the molecular structures for a C60-GLN-GABA synthesis 500. C60 510 is mixed with at least one glutamine (GLN) 520 and at least one gamma amino butyric acid (GABA) 530 under reactive shear mixing conditions at or below about 55° C. to form the reaction product as indicated in the direction of the downward pointing black arrow, which is C60-GLN-GABA. The GLN functional group 540 is carbonyl-pi-bonded to the C60 functional group 550 by the aromatic pi-carbonyl bonds indicated by dashed lines 560, 570. The GABA functional group 580 forms the aromatic pi-carbonyl bond indicated by dashed line 590. The resulting C60-GLU-GABA is already potent in establishing a more normal glutamine-glutamate cycle in the remediation of Alzheimer's symptoms, especially in those cases where there is sufficient ATP in the neural structures to promote energy impulses, however in many cases there may also be a comorbid energy deficit because of a lack of ATP. Therefore, the C60-GLN-GABA ensemble can be considered an intermediate to the synthesis of such a more robust nanoparticle, or it can also be an exemplary spalled or metabolized residual product of such a more robust nanoparticle ensemble, according to these teachings.

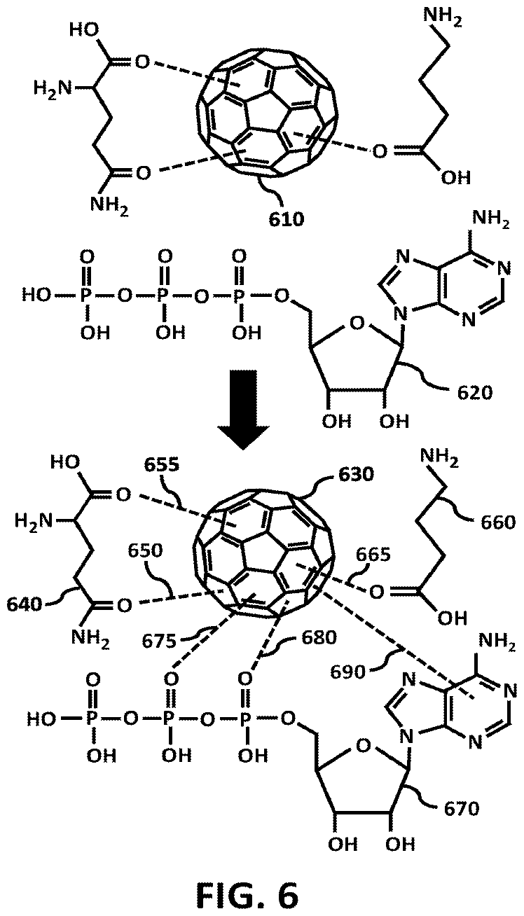

FIG. 6 illustrates the molecular structures for a C60-GLN-GABA-ATP synthesis. The intermediate reaction product C60-GLN-GABA 610 is combined with adenosine triphosphate (ATP) 620 under reactive shear mixing conditions at or below 55° C. to form the molecular product C60-GLN-GABA-ATP in which this reaction proceeds as indicated by the direction of the large black downward facing arrow. The produced nanoparticle ensemble consists of C60 630 which is aromatic pi-carbonyl bonded to GLN 640 as indicated by the dashed lines 650, 655, and is also aromatic pi-carbonyl bonded to GABA 660 as indicated by the dashed line 665. The ATP 670 may become bonded to C60 through any or all of its phosphate groups as indicated by the dashed lines for the exemplary two aromatic pi-phosphonyl bonds 675, 680, as well as by the aromatic pi-pi stacking bond between C60 and the adenine ring of ATP as indicated by the dashed line 690. The C60-GLN-GABA-ATP nanoparticle molecule is designed to be the most robust solution to treat the root causes of Alzheimer's disease pathology; it supplies the ATP neurotransmitter component to better promote biochemical energy availability in the human brain, while also providing the antioxidant functions of GABA and of C60, and simultaneously remediating the deficit in glutamine at the presynaptic terminals of neurons in the brain of Alzheimer's disease patients, according to these teachings.

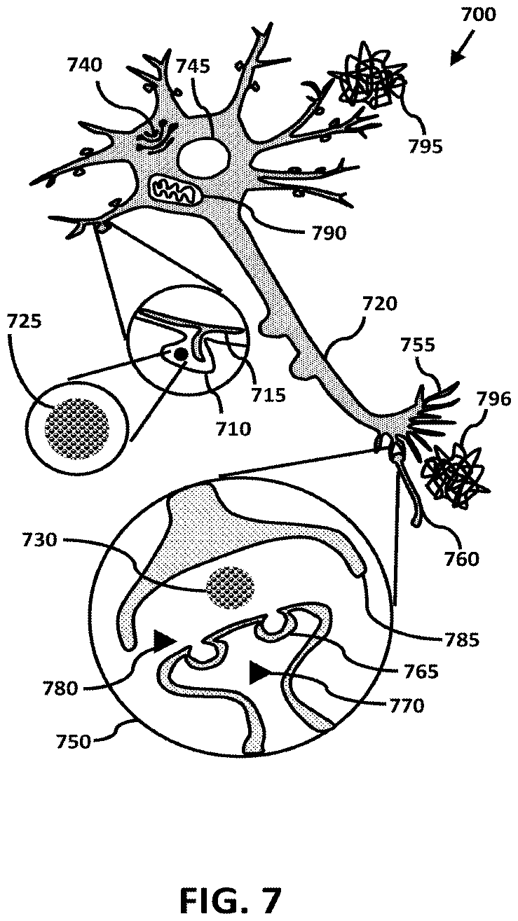

FIG. 7 illustrates neuronal cell 700. A dendrite 710 is illustrated in the circled expanded inset view; this view also illustrates endoplasmic reticulum (ER) 715 extending throughout the cell cytosol where it is bounded by the cell plasma membrane (PM) 720. The ER is in physical proximity with the plasma membrane to expedite lipid transfer, Ca2+ ion homeostasis, and synaptic plasticity. The nanoparticle ensemble of C60-GLN-GABA-ATP 725, 730 helps to shuttle anions and cations through the cytosol and across organelles and membranes. Vesicles originate at the Golgi apparatus 740 to transport lipids, calcium and other cations, hydrogen protons, electrons, and cellular signaling molecules. In Alzheimer's disease and other neuronal pathologies, effective transport of critical cellular materials from the cell nucleus 745 and the Golgi apparatus 740 via the ER 715 to the plasma membrane 720 can become compromised. The interposition of the neurotransmitter nanoparticle composition, C60-GLN-GABA-ATP can facilitate the transport of such cellular materials including electrons and protons between the ER 715 and the plasma membrane 720 to restore and remediate functional neuronal processes in neurons. Another expanded view 750 illustrates a synapse at the junction of a first neuron 755 and a second neuron 760. The presynaptic bouton 765 releases a multiplicity of internal neurotransmitters 770 into the synaptic cleft as free neurotransmitters 780, where the multiplicity of these neurotransmitters may include any of glutamine, GABA, and ATP molecules. The post synaptic terminal 785 accepts the ionic and electrical signals provided by the presynaptic terminal that are conveyed in part by the released neurotransmitters along with cations such as sodium. The glutamine functional group bonded to the C60 nanoparticle compound stabilizes conditionally essential glutamine to remediate local glutamine deficits in the synaptic cleft against upregulated astrocyte conversion of glutamine to glutamate, thereby therapeutically restoring synaptic glutamine homeostasis and reducing brain inflammation.

The nanoparticle ensemble of C60-GLN-GABA-ATP 730 helps to shuttle anions and cations across the synaptic cleft to accommodate deficits in bioenergetic signaling ability originating substantially in the loss of sufficient ionic and electrical charges arising from the neural mitochondria 790, thereby overcoming a loss of neurological signal strength. The normalization of blood pressure and the phosphate ion shuttling effect of the C60-GLN-GABA-ATP promotes the needed energetics to remediate sexual dysfunction, which is common in Alzheimer's and Lewy body disease patients. In males, this sexual dysfunction is termed erectile dysfunction; correction of this medical issue is short term, spontaneous, voluntary, and will become efficacious within 4 hours after administration.

Any of the neuron extracellular surfaces may become compromised to ion and neurotransmitter transport by the accumulation of salt-bridge stabilized oligomeric tangles of beta-amyloid and tau 795, 796 which also cause brain inflammation in Alzheimer's patients. These oligomeric structures can be disentangled by the abstraction of sodium and other cations by the C60 functional group of the nanoparticle ensemble, while the conditions of energy deficit and glutamine deficit are remediated by the ATP and GLN functional groups, and the anti-inflammatory properties of the C60 and GABA functional groups serve to promote a return to homeostatic and functional neurological conditions.

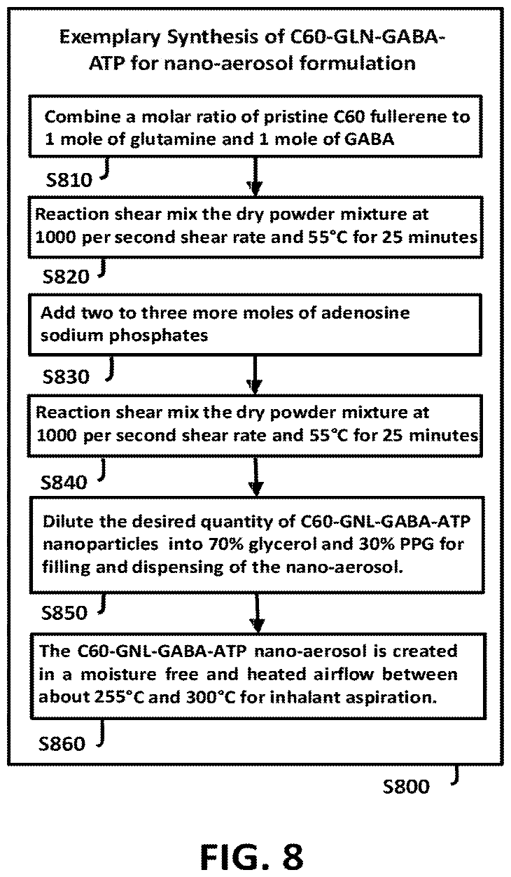

FIG. 8 is a flowchart representation of an exemplary scalable synthesis method S800 of C60-GLN-GABA-ATP formulated for nano-aerosol administration. In step S810, a molar ratio of vacuum sublimated C60 fullerene is combined with 1 mole of glutamine and 1 mole of GABA. In step S820 the prepared dry powder mixture is reaction shear milled for 25 minutes at about 55° C. to achieve the C60-GNL-GABA intermediate product. A shear pressure of about 20 grams per square micron is sufficient to create a slightly geometric oblate spheroid of the C60 molecule and simultaneously shift the density of states of the electrons of this cage molecule into anisotropic electrostatic distributions that achieve a metastable state when abutted to simultaneously induced opposing electrostatic charges with at least one abutting proximal GLN or GABA neurotransmitter. In step S830, two to three moles of adenosine triphosphate are added to the intermediate product. In step S840, the prepared dry powder mixture is reaction shear milled at about 55° C. for 25 minutes to achieve the C60-GNL-GABA-ATP final product. In step S850, the desired concentration of C60-GNL-GABA-ATP is created by dissolving a weighed amount of the dry powder into, for example, a 70% glycerol and 30% polypropylene glycol solvent mixture by volume. In step S860, a metered amount of the nano aerosol fluid from step S850 is generated by a commercially available electronic dispensing device suitable for personal inhalant aspiration by means of a heated airflow between about 255° C. and 300° C. to create the nano-aerosol; other methods of dispersing and inhaling this nano-aerosol formulation are possible and allowed, according to these teachings.

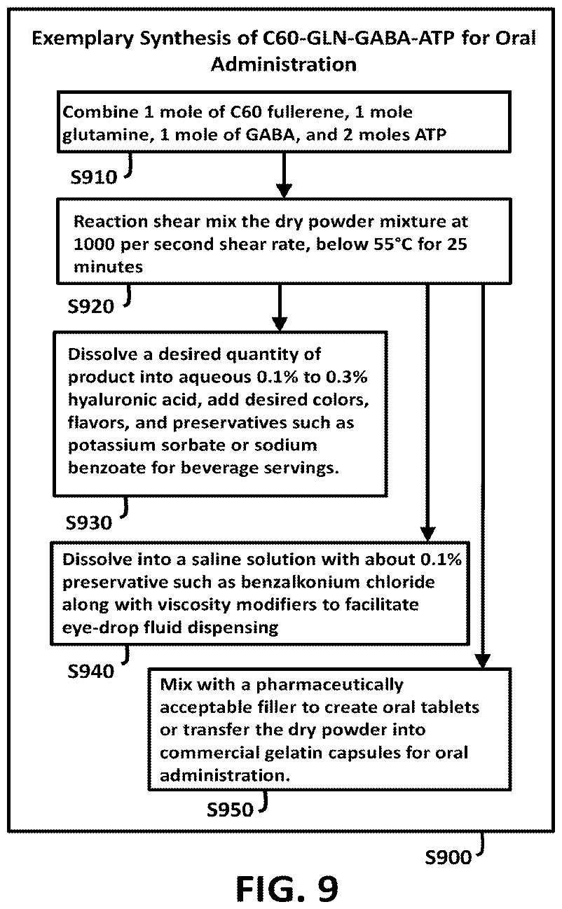

FIG. 9 illustrates a flowchart representation of an exemplary scalable synthesis method S900 for methods of oral administration. In step S910, 1 mole of C60 fullerene is combined with 1 mole glutamine, 1 mole of GABA, and 2 moles ATP. In step S920, the dry powder mixture is reaction shear mixed at 1000 per second shear rate, below 55° C. for 25 to 35 minutes or until reaction completion to produce C60-GNL-GABA-ATP as verified, for instance, by FTIR experimental analysis. In step S930, a desired quantity of product from step S920 is dissolved into aqueous 0.1% to 0.3% hyaluronic acid with stirring. Add desired colors, flavors, and preservatives such as potassium sorbate or sodium benzoate for beverage servings. Alternatively in step S940, a desired quantity of product from step S920 is dissolved into a saline solution with about 0.1% preservative such as benzalkonium chloride along with viscosity modifiers to facilitate eye-drop fluid dispensing. The human eye is one of the most rapid methods of bringing the dissolved C60-GNL-GABA-ATP into the human brain without passing through the lungs or the digestive system. Alternatively, in step S950, a desired quantity of product from step S920 is mixed with a pharmaceutically acceptable filler to create oral tablets or the dry powder is transferred into commercial gelatin capsules for oral administration. It is to be understood that other methods of oral administration are possible, for example the dry powder of C60-GNL-GABA-ATP obtained from step S920 may be directly added to a food or beverage using volumetric measuring spoons to transfer an appropriate amount for consumption.

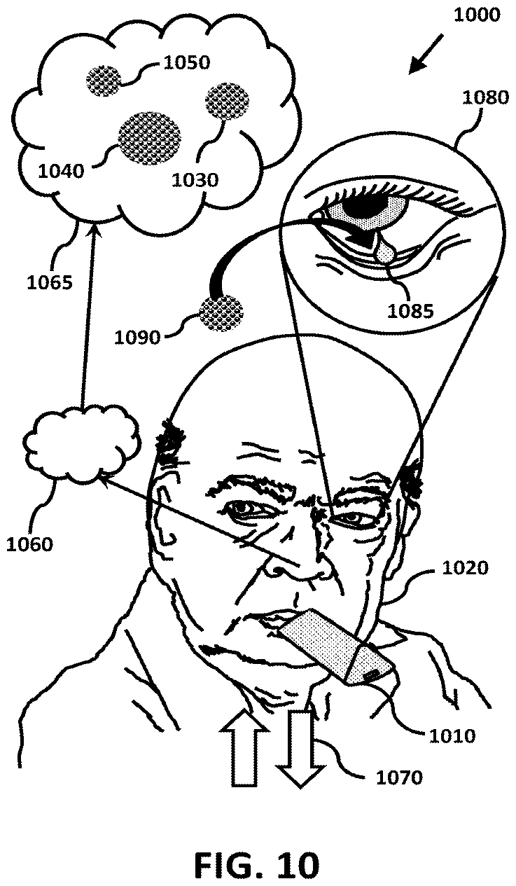

FIG. 10 illustrates a method 1000 for the personal administration of aspirated nano-aerosol delivery, and an ocular delivery method of C60-GNL-GABA-ATP. For the aspirated delivery method, the nano-aerosol generating device 1010 is filled with C60-GNL-GABA-ATP dispensing solution provided for dispersing and nebulizing of the inhalant gas including the nano-particles. The device 1010 may also be more commonly known as a nebulizer, or an electronic vaporizing device, or an electronic cigarette, or the functional part of a hookah to be shared among several users. In all cases these systems serve to carry the C60-GNL-GABA-ATP in a carrier fluid dispenser 1010, and to transfer that composition in nebulized form along with an aerosolized solvent in a substantially gaseous dispersion to the nose, mouth, trachea, and airways of a patient or user 1020. The use of the vaporized C60-GNL-GABA-ATP composition is to treat Alzheimer's disease or Lewy Body disease wherein the nano-aerosol can expedite targeted delivery to the brain by avoiding a passage through the digestive system. On arriving at the brain, the C60-GLN-GABA-ATP and its metabolites disrupts sodium ion salt bridges between beta amyloid plaque fibrils to return individual amyloid monomers to their proper conformation and neurological function, as well as to address the root causes of brain inflammation by providing antioxidant properties, a return of bioenergetics, and a stabilization of the glutamate-glutamic acid cycle.

Some of the nano-aerosolized composition is exhaled and shown as particulate clusters 1030, 1040, 1050 within exhaled smoke puffs 1060 and 1065 emitted on exhalation as indicated by the direction of thin line arrows radiating away from the nose of the subject. Systems that may be used for the method of dispersion of the C60-GNL-GABA-ATP represented by dispenser 1010, include, without limitation, any of the electronic cigarette devices produced internationally and listed in Appendix 4.1, “Major E-cigarette Manufacturers” of the “2016 Surgeon General's Report: E-Cigarette Use Among Youth and Young Adults” published by the Center for Disease Control and Prevention (CDC), Office of Smoking and Health (OSH) freely available at the CDC.GOV website, or any combination of piezoelectric, resistively heated, or inductively heated vaporized fluid delivery methods that can be utilized to deliver the composition of the present invention, especially when approved as a medical drug delivery device. Each embodied variation of such methods without limit are intended to aspirate aerosols as the method of therapeutic substance delivery of the composition of the present invention directed into the nasal cavities, mouth, tracheal breathing orifice, or intubated trachea of a patient. The supply direction of nebulized feed of C60-GNL-GABA-ATP on inhalation and exhalation are delivered into the airways and lungs of the intended patient by the flow of supplied air as indicated by the direction of upward and downward facing large white arrows 1070. An ocular delivery method to the consumer or patient 1020 is shown at the enlarged inset view of an eye, 1080, wherein viscosity stabilized saline eye drops 1085 are introduced. The eye drops 1085 are formulated to contain C60-GNL-GABA-ATP 1090 in a dispersed suspension or solution for comfort and rapid administration when other methods of administration are less effective for the patient or consumer being treated for Alzheimer's disease or Lewy Body disease.

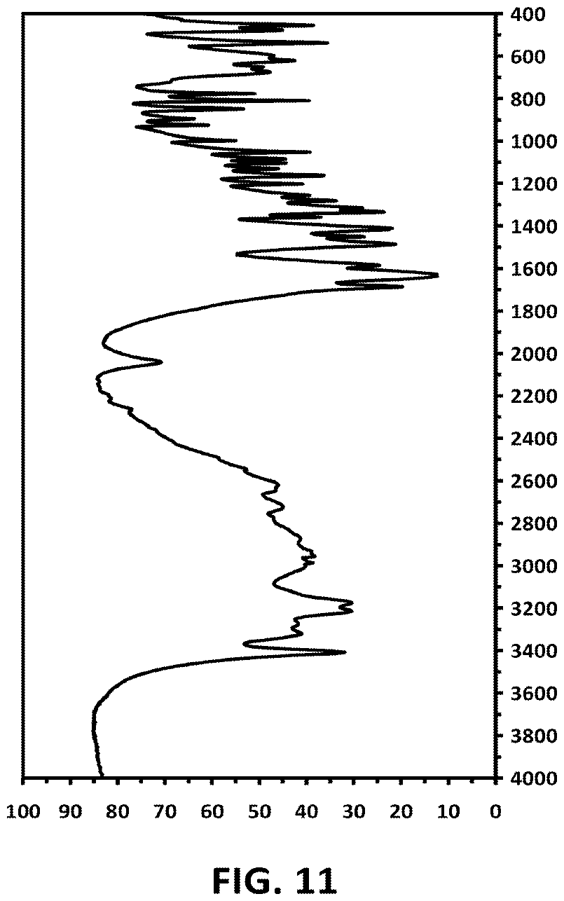

FIG. 11 illustrates experimental FTIR data for glutamine (GLN). All the Fourier transform infra-red (FTIR) spectrographs hereinafter were measured by transmittance using the potassium bromide (KBr) compressed flow solid pellet compact preparation method. The material used for analysis was obtained by the method of mixing, crushing, and consolidating under 7 metric tons of pressure, about 0.001 grams of the analyte substance with 1 gram of a diluent solid KBr that is substantially transparent to infrared light, and which flows under pressure to form a translucent pellet of about 0.4 mm thickness. Spectral background subtraction in air using a control pellet of the same mass and thickness having pure KBr was used to obtain a baseline instrument infrared spectral response. This method is generally referred to as the ‘KBr pellet’ sample preparation method, and it is used hereinafter throughout for each FTIR experimental data collection and spectral analysis. The Fourier transform infrared spectrophotometer used herein to obtain FTIR spectra throughout, is a model RF6000 FTIR instrument manufactured by Shimadzu of Japan. Each FTIR data graph hereinafter is provided with a numeric scale ranging from 400 to 4000 to represent reciprocal centimeters or (cm⁻¹) in wavenumbers.

The numeric scale ranging from 0 to 100 represents percentage transmittance and has units of %. The FTIR absorbance peak for glutamine (GLN) at stretching vibrations of the NH₃(+) group in hydrogen bonds are observed at 2717 cm⁻¹, 2623 cm⁻¹ and 2616 cm⁻¹, while the absorbance at 455 cm⁻¹ is attributed to the NH₃(+) torsional vibration mode. The uncharged primary amine (NH₂) absorbances appear at 3406 cm⁻¹, and 3215 cm⁻¹. Strong carbon hydrogen stretch (CH₂) absorptions appear at 3173 cm⁻¹ and 2931 cm⁻¹. The carbonyl stretching mode (C═O) is observed at 1629 cm⁻¹, and the disassociated carboxylic acid functional group, COO(−) has a stretching vibration at 1686 cm⁻¹. A primary amine scissoring absorption mode is observed at 1586 cm⁻¹. The overall band absorbances and peak assignments are in accordance with the FTIR spectra reported in published research journal reports for L-glutamine and serve to help interpret the chemical character of the experimental FTIR results obtained herein.

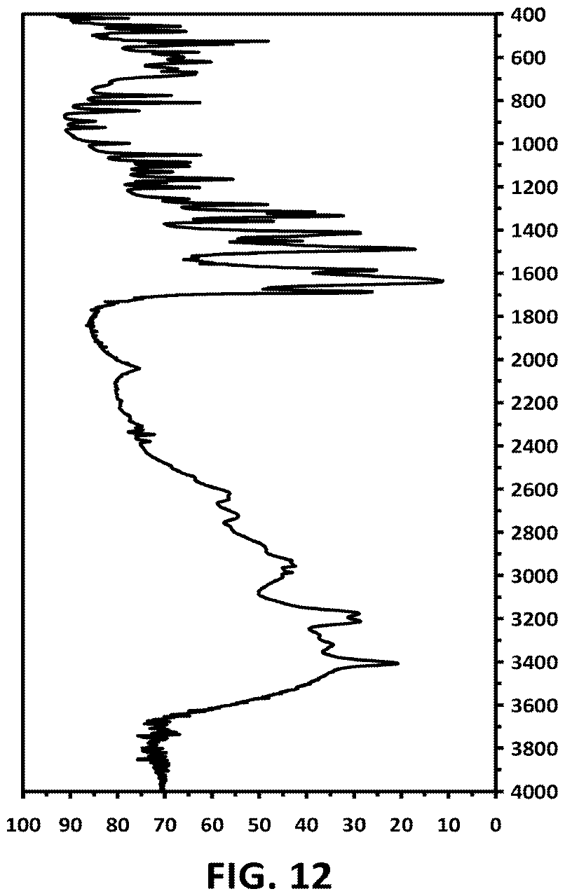

FIG. 12 illustrates experimental FTIR data for buckminsterfullerene glutamine (C60-GLN). The numeric scale ranging from 0 to 100 represents percentage transmittance and has units of %. The FTIR absorbances for C60-GLN have the expected stretching vibrations of the NH₃ (+) group in hydrogen bonds at 2718 cm⁻¹, 2629 cm⁻¹ and 2618 cm⁻¹, as well as the absorbance at 456 cm⁻¹ attributed to the NH₃(+) torsional vibration mode. An uncharged primary amine (NH₂) absorbance at 3406 cm⁻¹ remains unchanged in both GLN and C60-GLN, however there is a significant shift from the GLN absorbance at 3215 cm⁻¹ to that of 3210 cm⁻¹ in the present FTIR spectrum for C60-GLN. The strong carbon hydrogen stretch (CH₂) absorptions appear unaltered at 3173 cm⁻¹ and 2931 cm⁻¹. The carbonyl stretching mode (C═O) is observed at 1636 cm⁻¹ indicating a significant chemical shift from that of GLN previously observed at 1629 cm⁻¹ and is attributed to the formation of the aromatic pi-carbonyl bonds in C60-GLN. The disassociated carboxylic acid functional group, COO(−) has a stretching vibration at 1687 cm⁻¹ which is virtually identical to that of GLN and is not expected to change for C60-GLN. A primary amine scissoring absorption mode is observed at the C60-GLN absorption band at 1586 cm⁻¹ and is identical for that of GLN. The overall FTIR spectral absorptions observed are consistent with the creation of the C60-GLN molecule that is useful in the synthesis of the nanoparticle composition of the present invention and represents one of the likely metabolites.

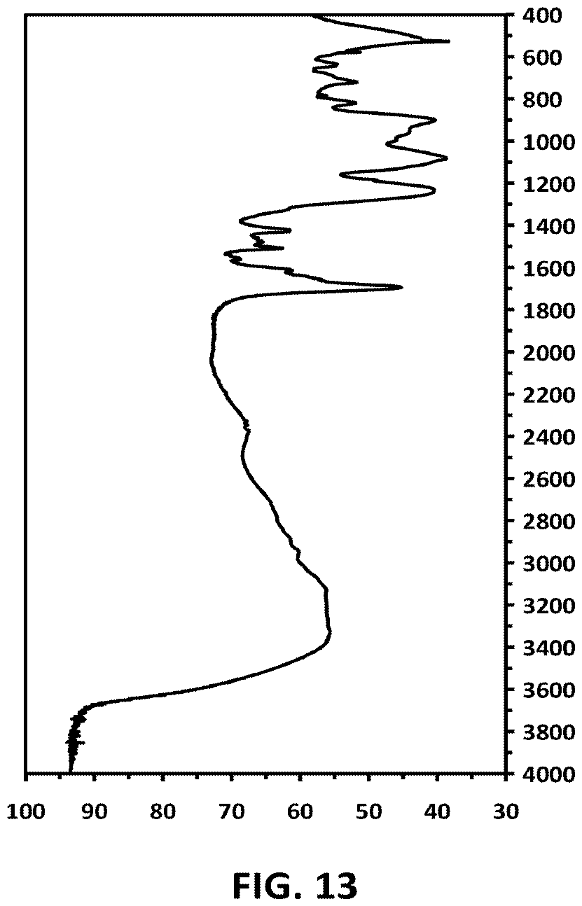

FIG. 13 illustrates experimental FTIR data for C60-ATP. The numeric scale ranging from 30 to 100 represents percentage transmittance and has units of %. The broad absorbance band from 3650 cm⁻¹ to 2600 cm⁻¹ is attributed to an additive combination of contributions from the hydroxyl groups of phosphates, and the ring nitrogen stretching vibrations from within the adenosine ring structure. The absorbance peak at 1694 cm⁻¹ is attributed to a vibration from double bonded phosphorus to oxygen (phosphonyl or P═O) functional groups. The easily recognized sharp C60 fullerene aromatic carbon-carbon stretching bands appear at 576 cm⁻¹ and 526 cm⁻¹. The absorbance at 1077 cm⁻¹ is attributed to carbon-oxygen ring vibrations in the adenosine functional group.

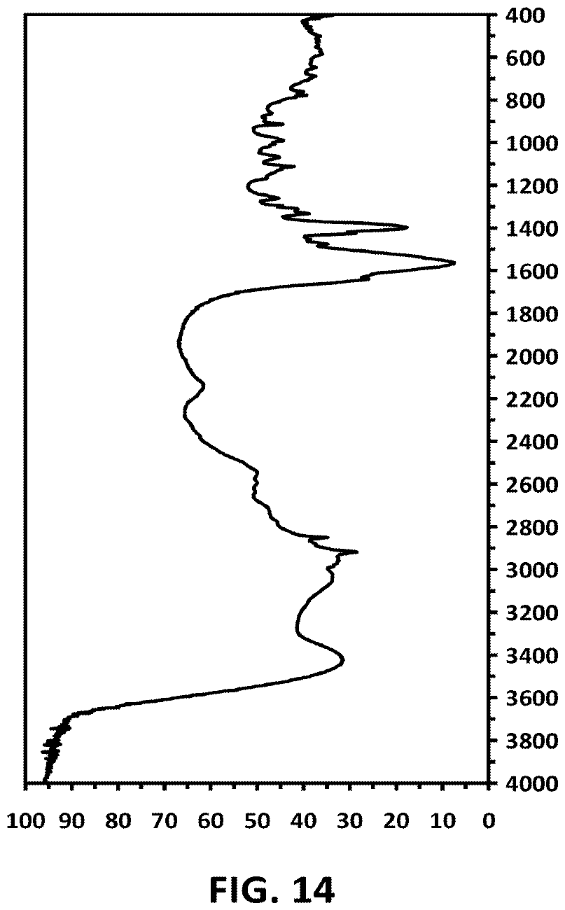

FIG. 14 illustrates experimental FTIR data for GABA raw material that was used to synthesize the compositions of the present invention. The numeric scale ranging from 0 to 100 represents percentage transmittance and has units of %. The FTIR absorbance peak at 3416 cm⁻¹ is attributed to the amine nitrogen-hydrogen vibration (N—H). The protonated amine group (NH3+) results in the observation of broad multiple peaks of the gamma aminobutyric acid spectrum in the 3300 cm⁻¹ to the 2600 cm⁻¹ range. It is notable the band at around 2125 cm⁻¹ has been associated with an amine hydrogen (N—H) stretching of zwitterionic salts. The absorption at 1563 cm⁻¹ indicates the presence of carboxyl functional group (C═O) symmetric stretching vibration that is characteristic of the GABA molecule. The strong and sharp peak observed at 1396 cm⁻¹ is attributed to a deprotonated oxygen as part of the carboxylic acid (COO—) in an asymmetric vibration mode of this functional group. This confirms the zwitterionic state of GABA by FTIR. The overall infrared absorbance spectral features are consistent with and indicate chemical similarity to GABA as may be found in published public FTIR spectra for this raw material.

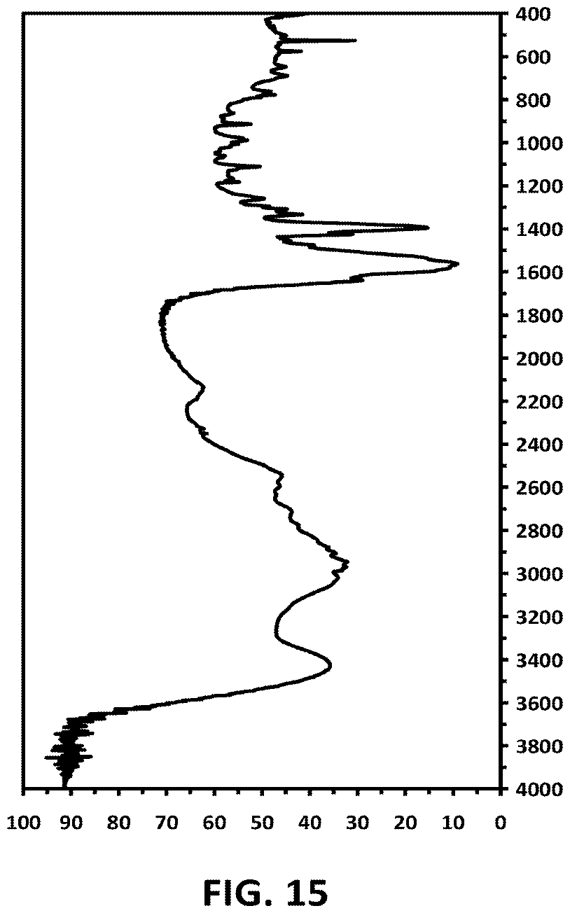

FIG. 15 illustrates experimental FTIR data for buckminsterfullerene gamma aminobutyric acid (C60-GABA). The numeric scale ranging from 0 to 100 represents percentage transmittance and has units of %. It is notable the band at around 2125 cm⁻¹ has been associated with an amine hydrogen (N—H) stretching of zwitterionic salts. A negatively charged ion or anion in this material is buckminsterfullerene (C60), which is known to accrue a charge of as many as six electrons. Also quite notable is the appearance of an enhanced absorption for protonated amine hydrogen (N—H) peak at 2946 cm⁻¹ in which this result supports the formation of a counter-ionic species that is reminiscent of the vibrational properties of a salt. It is also a characteristic of C60 to have a strong affinity to store protons as counter-charges. Therefore, the strengthening of the band at 2496 cm⁻¹ compared to the same region for GABA in FIG. 13 is attributed to the anionic C60 salt of GABA contribution. The absorption at 1563 cm⁻¹ indicates the presence of carboxyl functional group (C═O) symmetric stretching vibration has not changed this overall characteristic of the GABA molecule. The strong and sharp peak observed at 1396 cm⁻¹ is attributed to a deprotonated oxygen group as part of the carboxylic acid (COO—) group and remains as an asymmetric vibration mode of this functional group. These features are consistent with a zwitterionic C60 organic salt of GABA by FTIR as a distinguishable material having clearly recognizable chemical features.

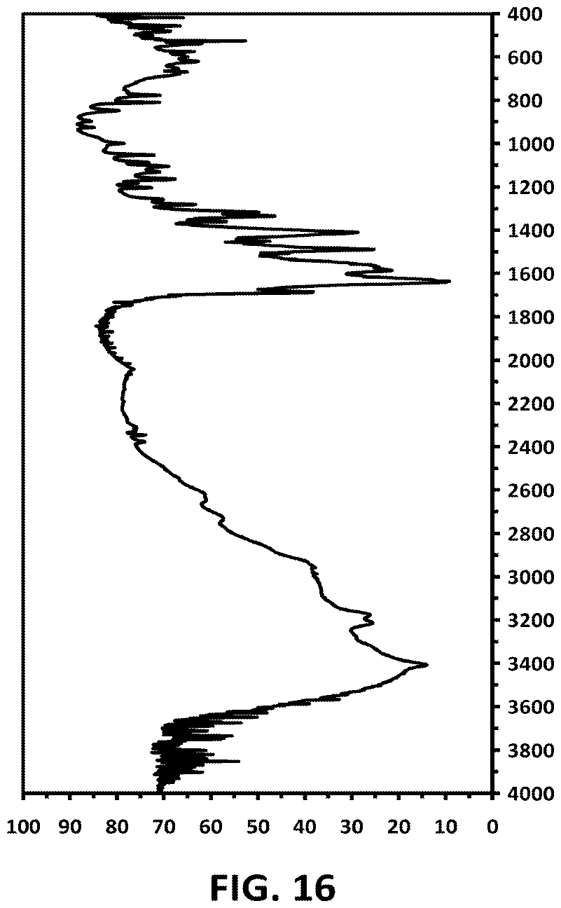

FIG. 16 illustrates experimental FTIR data for C60-GLN-GABA. The band at around associated with an amine hydrogen (N—H) stretching of zwitterionic salts has shifted from position for C60-GABA at 2125 cm⁻¹ to a new location at 2041 cm⁻¹, indicating the zwitterion is now placed into a chemically different environment. The characteristic sharp C60 fullerene aromatic carbon-carbon stretching bands appear at 576 cm⁻¹ and 526 cm⁻¹. While many of the observed absorption bands are unaltered from the contributions of both C60-GLN and C60-GABA, there are notable shifts in some bands that are attributed to chemical changes that do not appear in either of these precursor materials. For example, the carbonyl (C═O) absorbance at 1637 cm⁻¹ is representative of the unaltered C60-GLN carbonyl contribution. However, in present C60-GLN-GABA spectrum, the carbonyl absorbance at 1586 cm⁻¹ represents a considerable shift from that of 1563 cm⁻¹ previously observed for C60-GABA, indicating that the presence of GLN at C60-GLN-GABA has altered the pi-carbonyl vibration mode via crowding of functional groups onto the C60 molecule.

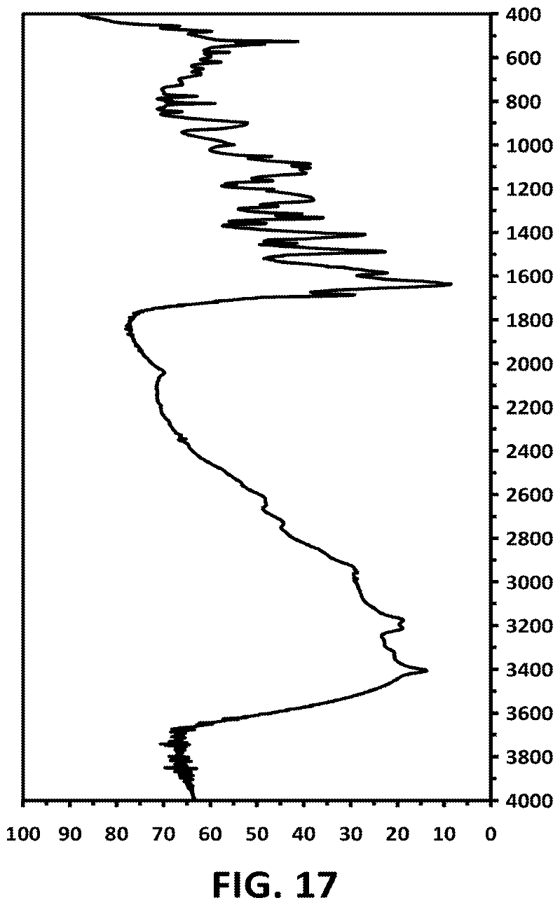

FIG. 17 illustrates experimental FTIR data for C60-GLN-GABA-ATP. The characteristic C60 fullerene aromatic carbon-carbon stretching bands appear at 576 cm⁻¹ and 526 cm⁻¹. One of the phosphate bands at 898 cm⁻¹ is likely conserved from a similar band observed for C60-ATP. The complex nature of this FTIR spectrum is chemically representative of the absorption patterns for the nanoparticle molecular ensemble of C60-GLN-GABA-ATP.

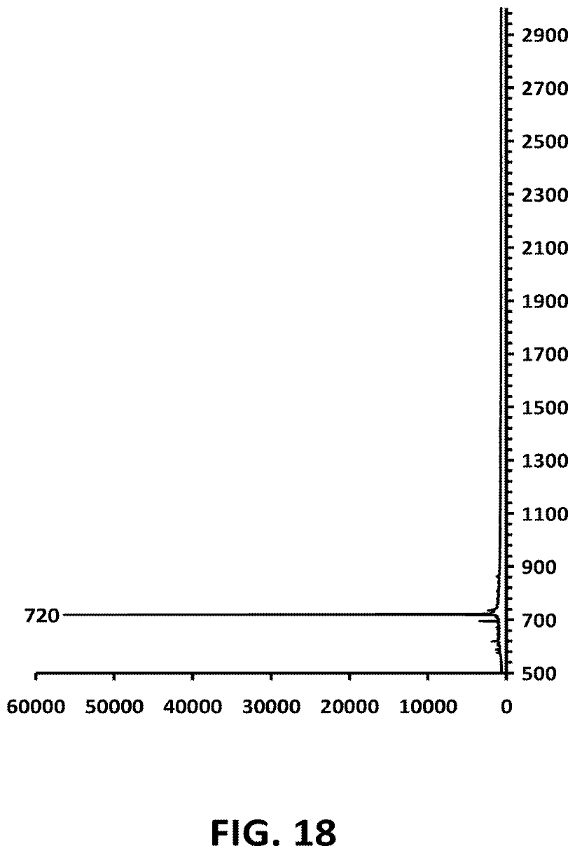

FIG. 18 illustrates the experimental negative mode mass spectrograph data for C60. This sample, as well as each of the subsequent MALDI-TOF experimental test results hereinafter, was introduced for test by laser vaporization into a Voyager Mass Spectrograph from Applied Biosystems (Foster City, California, USA). Negative mode bombardment was by fast moving electrons at about 70 eV energy. This resulted in molecular fragmentation and electron removal from the highest molecular orbital energy as molecular ions were formed. The ratio of mass to charge (m/z) is used to determine the molecular ion fragments to help determine the pieces of the original molecule in this assay. The mass peak at 720 m/z corresponds to the molecular ion fragment of fullerene C60 of mass 720.

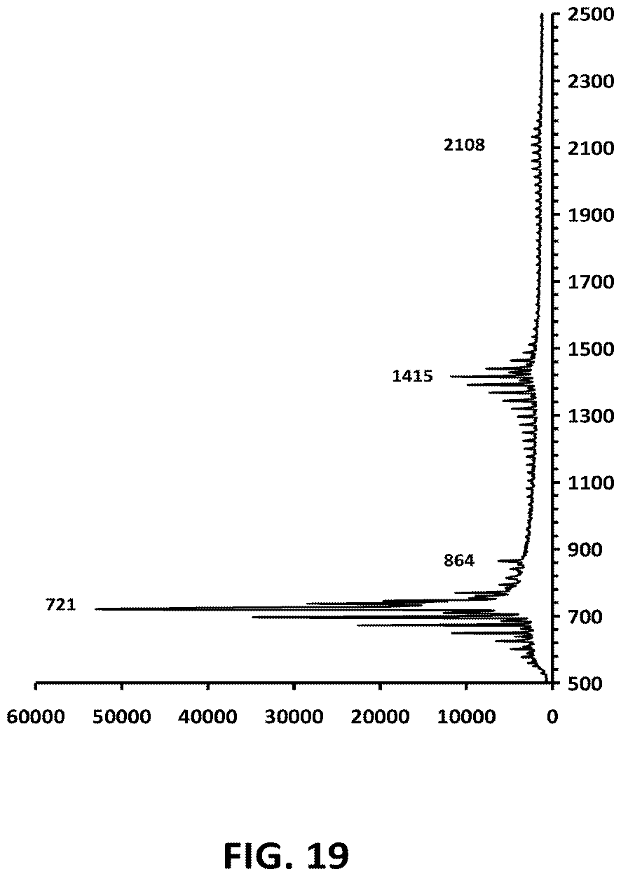

FIG. 19 illustrates experimental negative mode MALDI-TOF mass spectrograph data for C60-GLU-GABA-ATP material. The mass peak at 721 m/z corresponds to the molecular ion fragment of fullerene C60 of mass 720 having one adducted hydrogen atom. The observed peak shoulder having a molecular fragment at 864 m/z is characteristic for a fullerene C60 obtaining a residual spallation fragment from GABA that was incompletely removed. The cluster of peaks with a maximum at 1415 m/z is attributed to dimeric C60 with the interposition of some adducted residual molecular fragments. The presence of the trace peak clusters at 2108 m/z are evidence of trimeric network structures of C60 provided with bridging residual molecular fragment functional groups. The overall result serves as confirmation the GLN, GABA, and ATP components have indeed formed adducts with the C60 functional group.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A nanoparticle compound comprising:
a buckminsterfullerene C60 bonded to a glutamine, a gamma amino butyric acid, and an adenosine phosphate functional group.

2. The nanoparticle compound of claim 1 wherein the adenosine phosphate functional group comprises adenosine triphosphate.

3. A method of curing, treating, or prophylactically avoiding sexual dysfunction in a subject, comprising the step of:
administering to the subject an effective amount of a compound including a buckminsterfullerene C60 bonded to a glutamine, a gamma amino butyric acid, and an adenosine phosphate functional group.

4. The method of claim 3 wherein administering the compound comprises administering a composition containing the compound in a pharmaceutically acceptable carrier.

5. The method of claim 4 wherein an oral dosage comprises up to about 500 mg of the compound.

6. The method of claim 4 wherein administering the compound comprises intramuscular, intravenous, or subcutaneous administration in an amount of from about 0.1 mg/Kg to about 5 mg/Kg.

7. The method of claim 3 wherein the adenosine phosphate functional group comprises adenosine triphosphate.

8. A method of curing, treating, or prophylactically avoiding Alzheimer's disease or Lewy Body Disease and some types of brain dysfunction in Autism in a subject, comprising the step of:
administering to the subject an effective amount of a compound including a buckminsterfullerene C60 bonded to a glutamine, a gamma amino butyric acid, and an adenosine phosphate functional group.

9. The method of claim 8 wherein administering the compound comprises administering a composition containing the compound in a pharmaceutically acceptable carrier.

10. The method of claim 9 wherein an oral dosage comprises up to about 500 mg of the compound.

11. The method of claim 9 wherein administering the compound comprises intramuscular, intravenous, or subcutaneous administration in an amount of from about 0.1 mg/Kg to about 5 mg/Kg.

12. A method of making a compound including buckminsterfullerene C60 bonded to a glutamine, a gamma amino butyric acid, and also bonded to an adenosine phosphate functional group, the method comprising:
bonding the glutamine to the buckminsterfullerene; and
bonding the gamma amino butyric acid to the buckminsterfullerene; and
bonding the adenosine phosphate functional group to the buckminsterfullerene.

13. The method of claim 12 wherein bonding each of the glutamine, the gamma amino butyric acid, and the adenosine phosphate functional group to the buckminsterfullerene are performed at no more than 55° C.

14. The method of claim 12 wherein bonding each of the glutamine, the gamma amino butyric acid, and the adenosine phosphate functional group to the buckminsterfullerene is performed by reaction shear mixing.

15. The method of claim 12 wherein bonding the glutamine, the gamma amino butyric acid, and the adenosine phosphate functional group to the buckminsterfullerene are performed together.

16. The method of claim 12 further comprising combining the buckminsterfullerene bonded to the glutamine, the gamma amino butyric acid, and the adenosine phosphate functional group with a pharmaceutically acceptable carrier.

17. The method of claim 13 further comprising adding the buckminsterfullerene bonded to the glutamine, the gamma amino butyric acid, and the adenosine phosphate functional group to a mixture of glycerol and polypropylene glycol.

18. The method of claim 12 further comprising dissolving the buckminsterfullerene bonded to the glutamine, the gamma amino butyric acid, and the adenosine phosphate functional group into a hyaluronic acid solution.

19. The method of claim 12 wherein the adenosine phosphate functional group comprises adenosine triphosphate.
